(19) United States
(12) Patent Application Publication
(10) Pub. No.: US 2022/0160917 A1
(43) Pub. Date: May 26, 2022

(54) WORKFLOW MANAGEMENT SYSTEM RELATING TO STERILIZATION, INFORMATION PROCESSING APPARATUS, MANAGEMENT METHOD, AND COMPUTER-READABLE STORAGE MEDIUM

(71) Applicant: CANON KABUSHIKI KAISHA, Tokyo (JP)

(72) Inventors: Kazuhide Okuno, Shizuoka (JP); Motohiro Furusawa, Shizuoka (JP); Hiroki Kato, Shizuoka (JP)

(21) Appl. No.: 17/433,646

(22) PCT Filed: Feb. 19, 2020
(86) PCT No.: PCT/JP2020/006593

§ 371 Date: Aug. 25, 2021

(30) Foreign Application Priority Data
Feb. 27, 2019 (JP) 2019-034744

(51) Int. Cl. A61L 2/24; A61L 2/28

(52) U.S. Cl. CPC A61L 2/24; A61L 2202/14; A61L 2202/18; A61L 2/28

(57) ABSTRACT

A management system that manages a workflow relating to sterilization of a sterilization target is provided. The workflow includes a plurality of stages including a packaging stage in which a set is formed by packaging one or more of the sterilization targets and a first indicator, and the first indicator includes a discoloration area whose color changes according to an achievement degree of a sterilization process. The management system includes: management means for managing management information of the workflow including sterilization related information of the set, wherein the sterilization related information of the set includes a determination result of the achievement degree, and notification means for performing a notification that work related to the workflow is not in a predetermined state, or a notification for preventing work related to the workflow from entering a predetermined state.

### TECHNICAL FIELD

The present invention relates to a management technique of a workflow relating to a sterilizing process of sterilization targets.

### BACKGROUND ART

Sterilization processes are performed on sterilization targets such as medical instruments in a hospital. On this occasion, so-called sterilization indicators such as a chemical indicator (hereinafter, CI) and a biological indicator (hereinafter, BI are used in order to determine the achievement degree of the sterilization process, The CI includes a discoloration area whose color changes according to the achievement degree of a condition needed for the sterilization process using sterilizing agents (such as vapor or hydrogen peroxide). The achievement degree of the sterilization process can be determined by the color of the discoloration area of the CI after the sterilization process. The BI includes indicator bacteria that is highly resistive to the sterilizing agent, and the indicator bacteria die out according to the achievement degree of a condition needed for the sterilization process. The achievement degree of the sterilization process can be determined by, after the sterilization process, cultivating the BI and confirming the presence of the indicator bacteria. Japanese Patent No. 3414291 discloses a management apparatus that manages records of the sterilization process. According to Japanese Patent. No. 3414291, date and time of the process of sterilizing a sterilization target, the sterilization condition, the CI determination result are recorded.

In recent years, emphasis is placed on maintaining the quality of the sterilization process. Japanese Patent No. 3414291 discloses that the sterilization process is recorded and managed, but does not disclose a configuration for improving the quality of the sterilization process based on records of the sterilization process.

### SUMMARY OF INVENTION

According to an aspect of the present invention, a management system that manages a workflow relating to sterilization of a sterilization target is provided. The workflow includes a plurality of stages that includes at least one of a packaging stage in which a set is formed by packaging one or more of the sterilization targets and a first indicator, a sterilization stage of subjecting the set to a sterilization process by a sterilization processing apparatus, and a using stage of using the one or more of the sterilization targets included in the set by determining an achievement degree of the sterilization process performed on the set using the first indicator of the set, and the first indicator includes a discoloration area whose color changes according to the achievement degree of the sterilization process. The management system includes: management means for managing management information of the workflow including sterilization related information of the set that is associated with identification information of the set, wherein the sterilization related information of the set includes a determination result of the achievement degree of the sterilization process determined using the first indicator, and which of the stages of the workflow has been completed with respect to the set can be determined from sterilization related information of the set; input means for inputting the sterilization related information of the set; and notification means for performing, to a user based on the management information, a notification that work related to the workflow is not in a predetermined state, or a notification for preventing work related to the workflow from entering a predetermined state.

Further features of the present invention will become apparent from the following description of exemplary embodiments with reference to the attached drawings.

### BRIEF DESCRIPTION OF DRAWINGS

FIG. 1 is a diagram illustrating a sterilization workflow according to one embodiment.

FIG. 2 is a diagram illustrating a configuration of a management system of the sterilization workflow according to one embodiment.

FIG. 3 is a diagram illustrating a CI according to one embodiment.

FIG. 4 is a cross-sectional view of a measurement apparatus according to one embodiment.

FIG. 5 is a cross-sectional view of a measurement apparatus according to one embodiment.

FIG. 6 is a cross-sectional view of a measurement apparatus according to one embodiment.

FIG. 7 is a cross-sectional view of a measurement apparatus according to one embodiment.

FIGS. 8A to 8C are diagrams illustrating a master information table according to one embodiment.

FIG. 9 is a diagram illustrating a type determination table according to one embodiment.

FIG. 10 is a diagram illustrating an analysis result based on the master information table according to one embodiment.

FIGS. 11A and 11B are diagrams illustrating the master information table according to one embodiment.

FIGS. 12A to 12C are diagrams illustrating a job management table according to one embodiment.

FIG. 13 is a diagram illustrating an analysis result based on the job management table according to one embodiment.

FIG. 14 is a diagram illustrating the master information table according to one embodiment.

FIGS. 15A to 15C are diagrams illustrating the job management table according to one embodiment.

FIG. 16 is a diagram illustrating an analysis result based on the master information table and the job management table according to one embodiment.

FIG. 17 is a diagram illustrating a stock information table according to one embodiment.

FIG. 18 is a diagram illustrating an analysis result based on the stock information table according to one embodiment.

FIG. 19 is a diagram illustrating the master information table according to one embodiment.

FIG. 20 is a diagram illustrating an analysis result based on discard information according to one embodiment.

FIG. 21 is a diagram illustrating the job management table according to one embodiment.

FIGS. 22A and 22B are diagrams illustrating the master information table according to one embodiment.

FIGS. 23A and 23B are diagrams illustrating the master information table according to one embodiment.

FIG. 24 is a diagram illustrating an analysis result based on the master information table and the job management table according to one embodiment.

FIG. 25 is a block diagram of a management system according to one embodiment.

FIG. 26 is a block diagram of an information processing apparatus according to one embodiment.

### DESCRIPTION OF EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted.

### First Embodiment

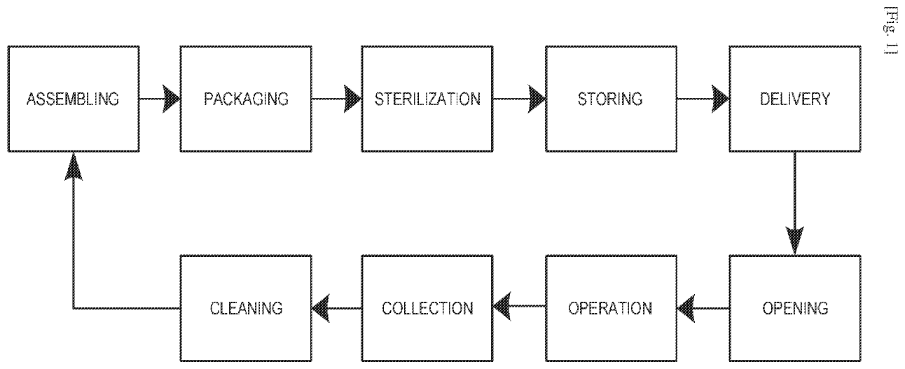

FIG. 1 shows a sterilization workflow relating to a sterilization processes. In the present embodiment, the sterilization workflow means a series of processes stages that are to be performed relating to a sterilization process with respect to a sterilization target. An assembly stage is a stage in which one or more sterilization targets are grouped in a sterilization station. Note that the number of sterilization targets included in one group may be one. The grouping may be performed based on a correspondence relationship between operations in which the sterilization targets are used and sterilization processing apparatuses that are used in sterilization processes of the sterilization targets. For example, the sterilization targets that are to be used in the same operation, and on which the sterilization processes are performed by the same sterilization processing apparatus may be grouped into the same group. A packaging stage is a stage in which one set is formed by packaging, in a sterilization station, one or more sterilization targets of one group and a CI (first indicator) using one packaging member. Usually, in hospitals or the like, a plurality of sterilization targets of the same type are used, and therefore a plurality of sets each including the same sterilization target(s) are formed. Hereinafter, each set including the same sterilization target(s) are referred to as a set of the same type. Conversely, the types of two sets being different means that at least one of the sterilization targets included in the respective two sets is different. The CI to be packaged into a set in the packaging stage is determined in accordance with the combination between the type of a set and the type of a sterilization processing apparatus that performs the sterilization process on the set, or the like. A sterilization stage is a stage in which a sterilization process is performed on a packaged set using a sterilization processing apparatus.

A storing stage is a stage in which a set on which the sterilization process has been performed in the sterilization stage is moved to and stored in a storage cabinet. A delivery stage is a stage of moving a set to be used in an operation or the like from the storage cabinet to a place of usage such as an operation room. An opening stage is a stage of opening the package of a set that has been moved to the place of usage. An operation stage is also a using stage that uses the sterilization target. Note that the achievement degree of the sterilization process is determined using the CI that is enclosed in the set before using the sterilization target in the opening stage, the operation stage, or both of the stages. Then, only when the achievement degree of the sterilization process can be determined to be sufficiently high using the CI, the sterilization target is actually used in the operation stage. A collection stage is a stage in which, after a sterilization target is used, the sterilization target is moved to the sterilization station. A cleaning stage is a stage in which a sterilization target is cleaned in the sterilization station. After a set is cleaned in the cleaning stage, the set is again transferred to the assembly stage. Note that the stages in the sterilization workflow in the present embodiment are merely examples, and the sterilization workflow of the present invention is not limited to the workflow including these stages. For example, the sterilization workflow may be a workflow that includes a stage other than those described above. Moreover, the sterilization workflow may be a workflow in which at least one of these stages is divided into a plurality of stages. Furthermore, the sterilization workflow may be a workflow in which at least two of these stages are integrated into one stage.

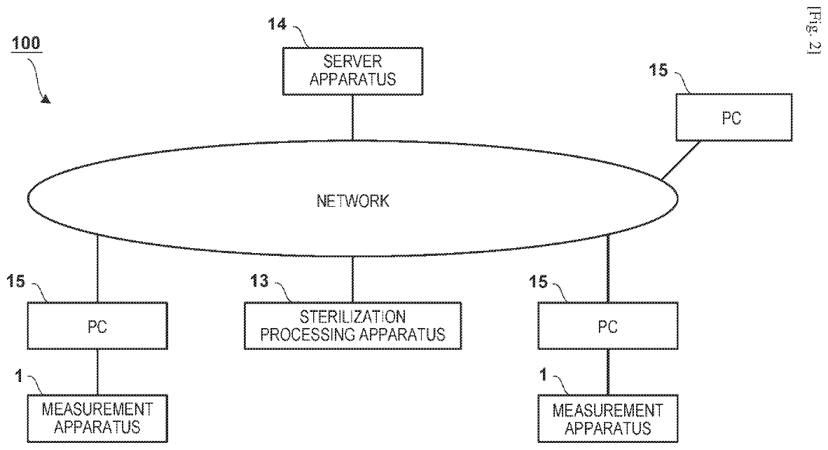

FIG. 2 is a diagram illustrating a configuration of a management system 100 that manages the sterilization workflow. A server apparatus 14, a plurality of personal computers (hereinafter, PCs) 15, and a sterilization processing apparatus 13 are connected to a network. The server apparatus 14, the PCs 15, and the sterilization processing apparatus 13 can communicate to each other via the network. The server apparatus 14 manages management information regarding the sterilization workflow. The PCs 15 can add information to the management information retained by the server apparatus 14, modify the retained information, and the like via the network. Also, the PCs 15 and the server apparatus 14 can notify a user of the management system 100 of various types of information based on the management information retained by the server apparatus 14. The notification of various types of information is performed through any method such as displaying the notification in a display of a PC 15, generating a sound from an unshown speaker, or transmitting a mail to an unshown PC that the user uses or a mobile terminal that the user has. As described above, although there are various methods regarding the manner with which various types of information is notified to a user, these are collectively referred to as “the management system 100 notifying (a user of)” in the following. Note that, in the present invention, the user means a person related to the sterilization workflow such as operators that actually perform works in the respective stages in the sterilization workflow, and a supervisor that supervises the operators.

The PCs 15 are installed at places where, in the sterilization workflow, management information needs to be referred to, information needs to be added to the management information, and the management information needs to be corrected. Also, as shown in FIG. 2, some PCs 15 are connected to respective measurement apparatuses 1. Each measurement apparatus 1 is an apparatus that measures a discoloration area of a CI. Note that, in the present embodiment, the measurement apparatus 1 is configured to also function as a reader apparatus that reads identification information of the CI in addition to the measurement of the discoloration area of the CI. The measurement apparatuses 1 are installed at places where a CI needs to be measured and places where the identification information of the CI needs to be read, and are each connected to a PC 15 installed at the same place. Note that the network in FIG. 2 is not limited to a closed network in a hospital. For example, the sterilization station may be located outside the hospital, and in this case, the network connects the hospital and the sterilization station outside the hospital. Also, the network may be a closed network such as a local area network (LAN) that is not connected to another network, or a network including the LAN and the Internet. For example, the network can be constituted by the Internet and a LAN inside a hospital to be connected the Internet, and in this case, the management system 100 can be configured by installing the server apparatus 14 outside the hospital and connecting it to the Internet.

Also, in the present embodiment, the sterilization processing apparatus 13 is connected to the network, and the PCs 15 and the server apparatus 14 can acquire information to be added to the management information and information for modifying the management information from the sterilization processing apparatus 13. However, the configuration may also be such that the sterilization processing apparatus 13 is not connected to the network. In this case, an operator performs addition, modification, or the like of the management information by operating a PC 15 based on information output from the sterilization processing apparatus 13. Note that, in order for an operator to perform input, update, display, or the like of the management information, the operator needs to operate a PC 15, the description of operating a PC 15 will be omitted in the following in order to simplify the description. Also, an operator inputting information to a PC 15 is also represented as an operator inputting information to the management system 100. Moreover, the management system 100 recording input information as a result of an operator inputting information to a PC 15 is also represented as an operator recording information to the management system 100. Furthermore, information to be presented to an operator being displayed in a display of a PC 15, being output to a printer connected to the PC 15, and being recorded by being output to a hard disk or the like of the PC 15 is also represented as the management system 100 presenting/outputting information (to an operator).

In the present embodiment, the sets are each given identification information. The identification information may be in the form of a serial number or a bar code that is printed on a seal, for example. Alternatively, the identification information may be recorded in an IC tag. A medium including the identification information may be attached to an exterior face of a packaging member of the set, or a sterilization target of the set, for example. Alternatively, the medium may be attached to a CI enclosed in the set. Also, when a serial number or the like is recorded in a CI itself, the serial number of the CI to be enclosed in a set can be used as the identification information of the set. In the present embodiment, the serial number of a CI enclosed in a set is used as the identification information of the set.

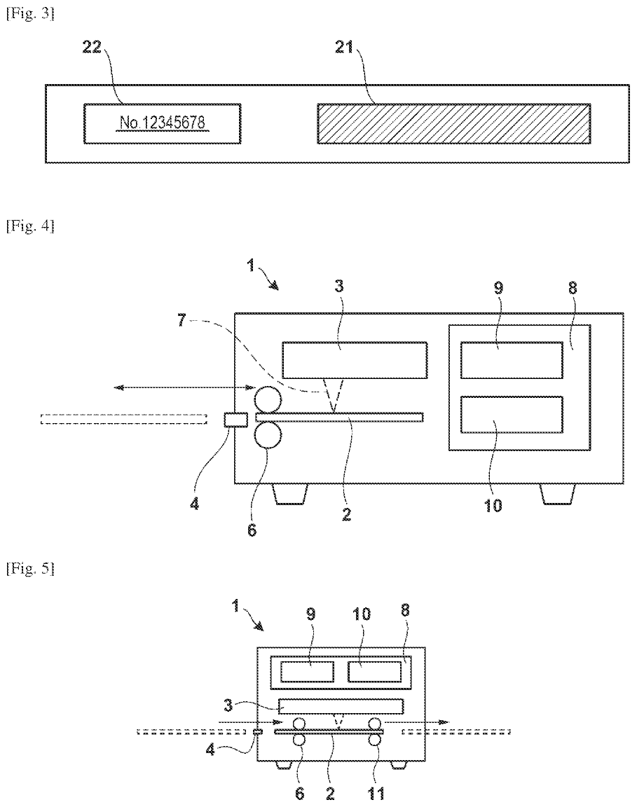

FIG. 3 shows a CI according to the present embodiment. The CI is a sheet-like test paper and includes a discoloration area 21 in which a chemical treatment is performed on its surface. The discoloration area 21 changes its color according to the achievement degree of a sterilization process. For example, the discoloration area 21 of a CI is configured such that its color value is a color value C#1 in an initial state, changes to a color value C#2, . . . , and to a color value C#9 according to the achievement degree of the sterilization process, and finally stabilizes at a color value C#10. Also, the CI includes an identification portion 22 having the identification information. In FIG. 3, the identification information is a serial number. Note that the identification portion 22 may also be a bar code printed thereon or an IC tag (wireless tag) attached to the CI, as described above. Also, in the present embodiment, it is assumed that the type of a CI can be determined from identification information of the identification portion 22. For example, when there are two types of CIs, namely T1 and T2, the first number of the serial number may indicate the type of the CI.

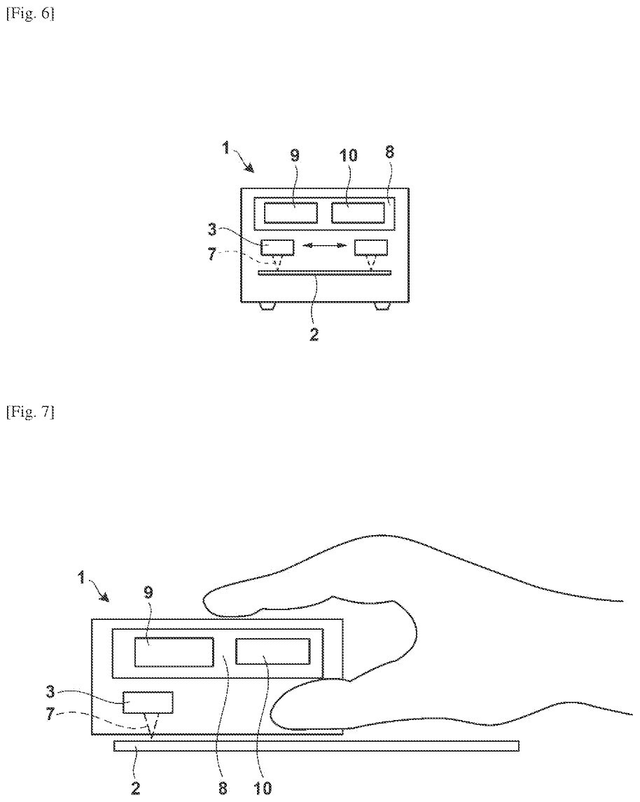

FIG. 4 is a cross-sectional view illustrating an example of the measurement apparatus 1. Note that the reference sign 2 in FIG. 4 indicates a CI that has been conveyed to a measurement position of the measurement apparatus 1, and the dotted line indicates a CI before being inserted into the measurement apparatus 1. The CI that has been inserted into an insertion portion 4 of the measurement apparatus 1 is conveyed to the measurement position by a conveyance roller pair 6. A measurement unit 3 measures the color of the discoloration area 21 of a CI. After the measurement of a CI, the CI is discharged outside the measurement apparatus 1 by rotating the conveyance roller pair 6 in a reverse direction. The measurement unit 3 reads an optical measurement value relating to the color value of the discoloration area 21 of a CI, e.g. spectral reflectance, using light 7. In the present embodiment, the measurement unit 3 is configured to read the identification information of a CI in addition to the color of the discoloration area 21 of a CI. If the identification portion 22 includes a serial number or a bar code, the measurement unit 3 can optically read the identification portion 22 of a CI. Note that if the identification portion 22 is an IC tag, the measurement unit 3 has an IC reader, and reads the identification information stored in the IC tag by communicating with the IC tag. Also, the measurement unit 3 can be configured to read the color value of the discoloration area 21 and the identification information by capturing an image of the discoloration area 21 and the identification portion 22 using an image sensor. A controller 8 controls the measurement apparatus 1. Various types of information to be used in the measurement are stored in a storage unit 9 of the controller 8. Also, a computational processing unit 10 of the controller 8 determines the color value of the discoloration area 21 of a CI based on a measurement result of the measurement unit 3, that is, spectral reflectance, for example. The controller S outputs the color information indicating the determined color value and the read identification information of a CI to a PC 15.

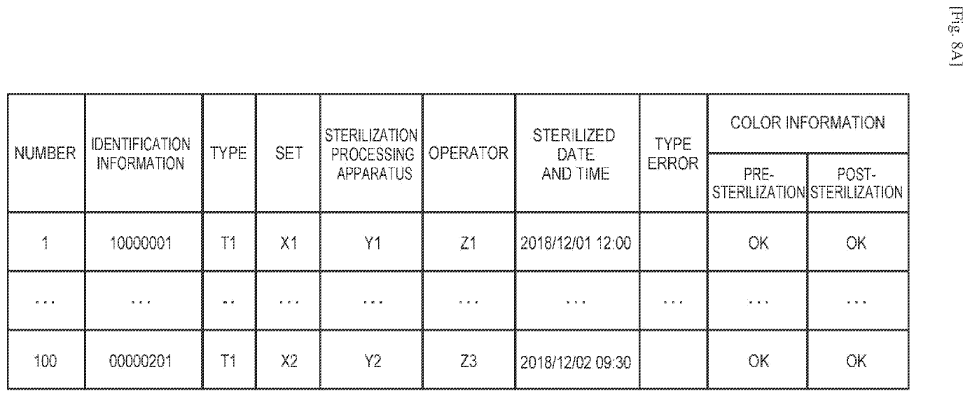

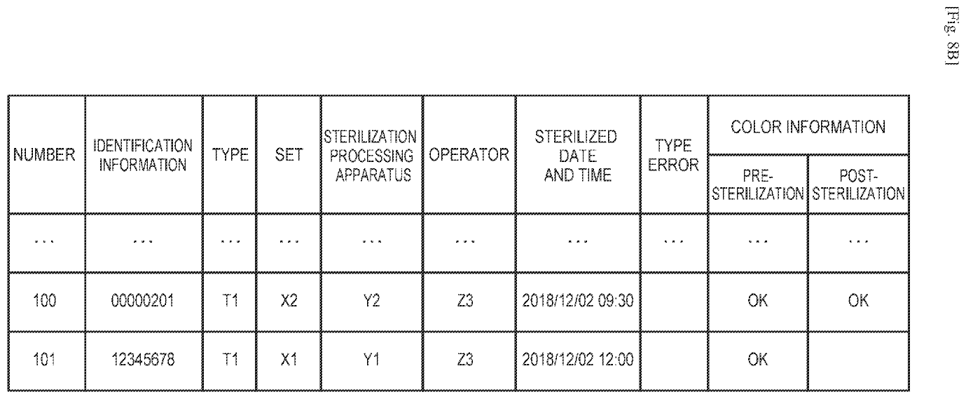

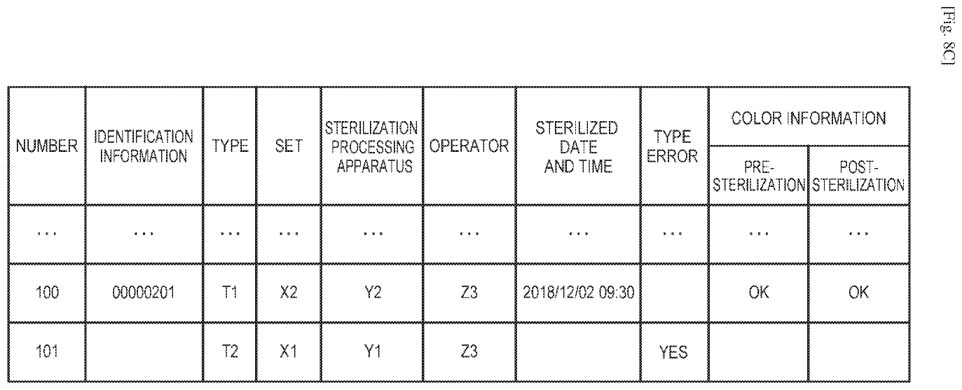

Note that the measurement apparatus 1 shown in FIG. 4 discharges a CI from the insertion portion 4, but the configuration of the measurement apparatus 1 may be such that, as shown in FIG. 5, a discharging portion is provided on an opposite side of the insertion portion 4 relative to the measurement position of a CI, and the CI is discharged from the discharging portion after measurement. Also, the measurement apparatus 1 shown in FIG. 4 is configured to measure, after a CI has been conveyed to the measurement position, the CI by a fixed measurement unit 3, but the measurement apparatus 1 may be configured such that, as shown in FIG. 6, the measurement unit 3 can be moved. That is, the configuration may also be such that the discoloration area 21 and the identification portion 22 of a CI are read by moving the measurement unit 3 in a predetermined direction. Moreover, as shown in FIG. 7, the configuration may also be such that the discoloration area 21 and the identification portion 22 of a CI are read by an operator moving the measurement apparatus 1 on the CI. Such a configuration is advantageous when CIs having various sizes are used.

Note that, when the identification information of a CI is not used as the identification information of a set, the identification information is read in accordance with a medium in which the identification information is recorded and a format of the identification information. For example, if the identification information is a bar code, a barcode reader can be used for reading the identification information. Also, if the identification information is a serial number, a configuration may also be adopted in which an operator inputs the identification information to a PC 15.

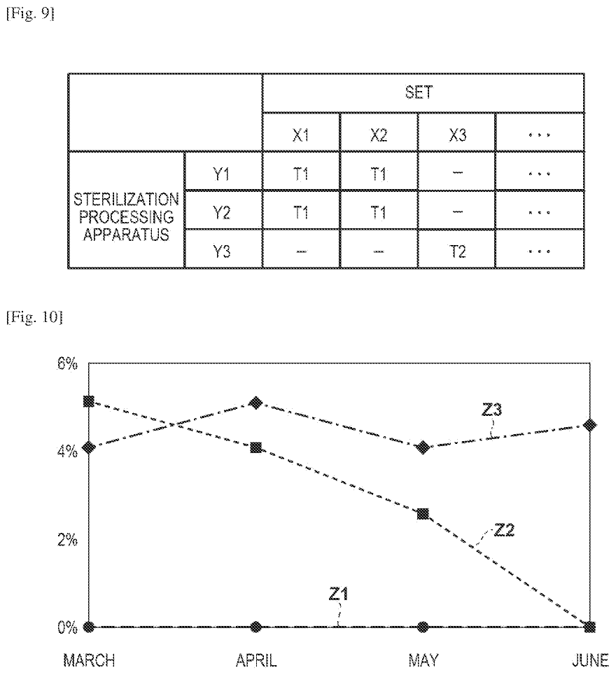

FIGS. 8A to 8C show a master information table, which is one of the pieces of management information retained in the server apparatus 14 in the present embodiment. A number field shows a number of each record. The record is provided with respect to each identification information of a set. The identification information of the set is stored in an identification information field. In the present embodiment, the identification information of a set is identification information of a CI enclosed in the set. Information indicating the type of a CI that is specified by the identification information is stored in a type field. Information for specifying the type of a corresponding set is stored in a set field. Information for specifying the type of a sterilization processing apparatus 13 that performs sterilization of the set is stored in a sterilization processing apparatus field. Information for specifying an operator that performs the sterilization process is stored in an operator field. Information for specifying the date and time at which the sterilization process has been performed is stored in a sterilization data and time field. Information indicating whether or not an operator has missed the type of a CI is stored in a type error field. A color information field includes two subfields of pre-sterilization and post-sterilization. In the present embodiment, the discoloration area 21 of a CI before being used in the sterilization process is measured by the measurement apparatus 1, and whether or not the CI has an initial failure is determined based on the measurement result. Also, the determination result is stored in a pre-sterilization subfield. Also, the discoloration area 21 of a CI on which the sterilization process has been performed is measured by the measurement apparatus 1, and whether or not the sterilization process has reached a predetermined achievement degree is determined based on the measurement result. Also, the determination result is stored in a post-sterilization subfield. In the following, the information of each field recorded in each record is also referred to as sterilization related information of a set.

For example, assume that the packaging stage is executed when the contents of the master information table are as shown in FIG. 8A. Here, an operator adds a new record (number 101) to the master information table as a recording target record by operating the management system 100. Also, the operator inputs, to the management system 100, information for specifying the type of a target set of the packaging stage, information for specifying the type of the sterilization processing apparatus to be used, and information for specifying the operator. With this, these pieces of information are respectively recorded in the set field, the sterilization processing apparatus field, and the operator field of the recording target record. On this occasion, the management system 100 displays the type of a CI to be used to the operator using a later-described type determination table. The operator causes the measurement apparatus 1 to read a CI of the displayed type. Here, it is assumed that the measurement apparatus 1 has read a CI whose identification information is 12345678 shown in FIG. 3, and the measurement apparatus 1 has output the color value (hereinafter, measured color value) Ct of the discoloration area 21 as the measurement result and identification information 123456678 to a PC 15. The PC 15, after confirming that the record corresponding to the identification information 12345678 is not present in the master information table, records the identification information 12345678 in an identification information field of the recording target record.

Also, the PC 15 determines whether or not the color difference between the color value C#1, which is an initial color of the discoloration area 21 of the CI, and a measured color value Ct exceeds a threshold value. If the color difference between the color value C#1 and the measured color value Ct does not exceed the threshold value, the PC 15 records “OK” in the pre-sterilization subfield. Note that if the color difference between the color value C#1 and the measured color value Ct exceeds the threshold value, the PC 15 records “NG” in the pre-sterilization subfield. At the same time, the PC 15 performs display indicating whether or not the color difference between the color value C#1 and the measured color value Ct exceeds the threshold value. Also, if the color difference between the color value C#1 and the measured color value Ct exceeds the threshold value, the management system 100 can display, to the user, a fact that the CI cannot be used in later stages. The operator determines, based on the information displayed in a display unit of the PC 15, whether the measured CI is to be enclosed in a set, or the CI is discarded and a different CI is to be measured.

FIG. 8B shows a state in which the CI enclosed in the set corresponding to the record of the number 101 added as described above does not have an initial failure, and the sterilization process has been thereafter performed on the set. The date and time at which the sterilization process has been performed is recorded in the sterilization data and time field, When the contents of the master information table are as shown in FIG. 8B, the set including the CI whose identification information is 12345678 is to be used in the operation stage. In this case, the CI is measured by the measurement apparatus 1. With this, the measured color value CI and the identification information 12345678 are output to the PC 15. The PC 15 searches the recording target record corresponding to the identification information 12345678 over the master information table, and confirms that the number of the recording target record is one. Next, the PC 15 determines the color, of the plurality of color value C#1 to C#10 that the discoloration area 21 can take, whose color difference from the measured color value Ct is the minimum. Then, if the color whose color difference from the measured color value Ct is the minimum is one of the color values C#8 to C#10, for example, the PC 15 determines that the target achievement degree has been reached (OK), and if not, determines that target achievement degree has not been reached (NG). The PC 15 records the determination result in the post-sterilization subfield of the recording target record along with displaying the determination result to the user.

For example, the set corresponding to a record in which data is recorded in only the identification information, type, set, and sterilization processing apparatus fields and the pre-sterilization subfield, is a set in a state after the packaging stage and before the sterilization stage. Moreover, the set corresponding to a record in which data is added in the sterilization data and time field is a set in a state after the sterilization stage and before the operation stage (before the using stage). Furthermore, the set corresponding to a record in which data is recorded in the post-sterilization subfield of the color information field is a set in a state after the operation stage (after the using stage). In this way, it can be determined which of the packaging stage, the sterilization stage, and the operation stage has been completed with respect to a set based on the sterilization related information.

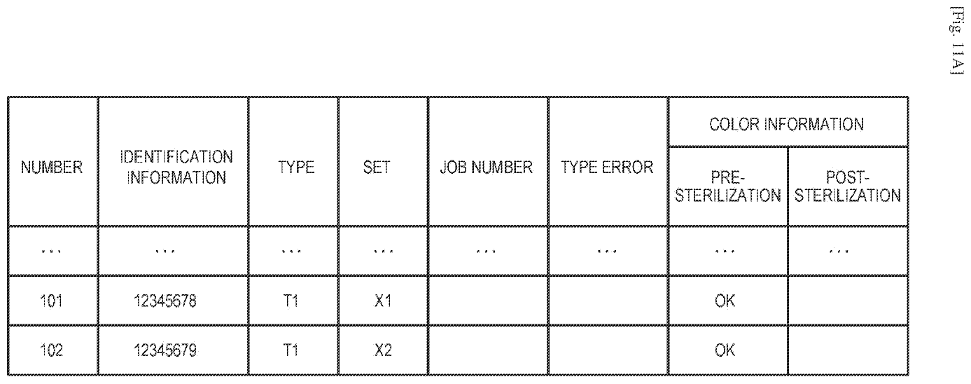

Here, if an operator has performed an action that is different from the required action in each stage, that is, the operator has performed a wrong work, the management system 100 notifies the operator of the fact. For example, if an operator tries to enclose a CI of a type that should not be used in the packaging target set in the packaging stage, the management system 100 notifies the operator of a type error of the CI based on the type determination table. FIG. 9 shows a type determination table retained by the server apparatus 14. The type determination table (type determination information) shown in FIG. 9 shows the type of a CI that should be used for a combination between the type of the sterilization processing apparatus 13 and the type of the set. According to FIG. 9, when the set of a type X1 is subjected to the sterilization process using the sterilization processing apparatus 13 of a type Y1, it is stipulated that a CI of a type T1 is to be used, for example. Also, according to FIG. 9, when the set of a type X3 is subjected to the sterilization process using the sterilization processing apparatus 13 of a type Y3, it is stipulated that a CI of a type T2 is to be used. Also, according to FIG. 9, it is also shown that a set of a type X2 cannot be subjected to the sterilization process using a sterilization processing apparatus of the type Y3.

For example, it is assumed that an operator has recorded X1 in the set field and Y1 in the sterilization processing apparatus field, in the packaging stage. The management system 100 displays a message saying that a CI of the type T1 should be used in the display unit based on the type determination table. However, if the operator has mistakenly caused the measurement apparatus 1 to read a CI of the type T2, the management system 100 records “YES” in the type error field, as shown in FIG. 8C. Also, the management system 100 notifies the operator of the type error of the CI. In this way, as a result of determining the type of a CI that should be used based on the type determination table, the operator can be prevented from using a CI of a wrong type.

Also, in order to maintain the quality of the sterilization workflow, the frequency of wrong actions by an operator needs to be reduced. For example, in the present embodiment, the fact that a CI of a wrong type has been read by the measurement apparatus 1 is also recorded in the master information table, and therefore a measure for improvement can be taken such that wrong determination regarding the type of a CI will not occur based on the records accumulated in the master information table.

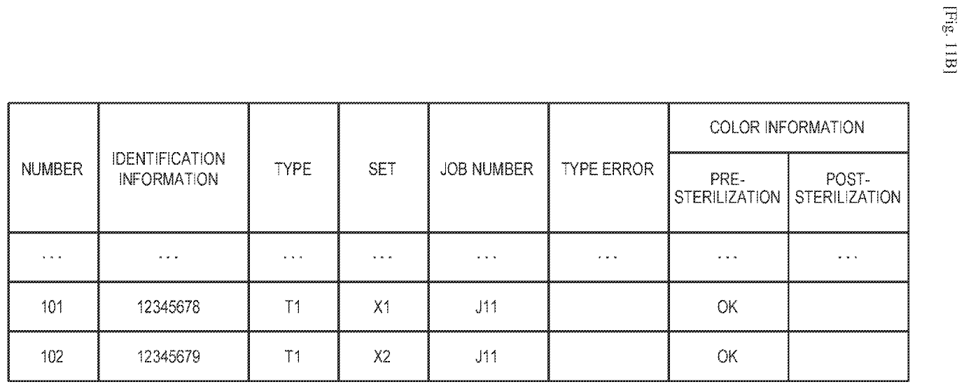

FIG. 10 shows an example of a graph that the management system 100 creates based on the master information table, and is presented to a user. The graph shown in FIG. 10 shows a monthly type error rate for each operator. The type error rate can be obtained by extracting monthly records for each operator, and calculating a ratio of the number of records in which “YES” is recorded in the type error field relative to the number of extracted records. According to FIG. 10, the type error rate of an operator Z1 is 0. Also, the type error rate of an operator Z2 is high in March, gradually decreases, and becomes 0 in June. On the other hand, the type error rate of an operator Z3 remains high.

From FIG. 10, it can be seen that, with respect to the operator Z2, the work in the packaging stage has improved, but with respect to the operator Z3, there is no improvement in the work. From the graph shown in FIG. 10, it can be understood that it is necessary that the operator Z3 is forced to recognize that the CI displayed in the PC 15 should be used, and training is performed on the operator Z3 with respect to the way how the types of CIs are distinguished in order to reduce the type error rate.

Note that, in the present embodiment, the type error of a CI to be used is used as an example of the work error, but the present invention is not limited thereto. For example, a configuration may be adopted in which an action of starting the operation of the sterilization process being not performed to the sterilization processing apparatus 13 in the sterilization stage, a wrong program that is used in the sterilization processing apparatus 13, and the like are detected as a work error and reported. Note that the information needed for detecting these errors is recorded as sterilization related information. Also, in the present embodiment, when the determination is performed based on the type determination table, the type error of a CI is determined assuming that data in the set field and the sterilization processing apparatus field input by the operator is correct. However, the configuration may also be such that, if the combination of entries in the type field, the set field, and the sterilization processing apparatus field that have been input by an operator does not match the combination shown by the type determination table, the operator is notified of the fact.

Note that, in the present embodiment and the following embodiments, the type of the sterilization processing apparatus 13 is recorded in the sterilization processing apparatus field. However, the configuration may also be such that information for specifying an individual sterilization processing apparatus 13 is recorded in the sterilization processing apparatus field instead of, or in addition to the type of the sterilization processing apparatus 13.

### Second Embodiment

Next, a second embodiment will be described focusing on the difference from the first embodiment. In the present embodiment, in the sterilization stage, a plurality of sets are arranged in a sterilization processing apparatus 13, and the plurality of sets are subjected to the sterilization process at the same time. In the following, the task in which a plurality of sets are subjected to the sterilization process at the same time using the sterilization processing apparatus 13 is referred to as a job. In the present embodiment, jobs are managed, and the relationship between sets and jobs is recorded. Also, in the present embodiment, a BI, which is a second indicator, and a CI are arranged at predetermined positions in the sterilization processing apparatus 13. The CI, which is a second indicator, is arranged inside a process challenge device, which is a so-called PCD, that is arranged in the sterilization processing apparatus 13, for example.

After the sterilization stage, a plurality of sets subjected to the sterilization process are stored in a storage cabinet, in the storing stage. On the other hand, the BI and the CI (hereinafter, PCD CI), which is a second indicator, that were arranged in the sterilization processing apparatus 13, are subjected to an indicator testing stage in which the BI and the CI are tested. The indicator testing stage is a stage in which the achievement degree of the sterilization process is determined using the BI and the PCD CI. If it is determined, in the indicator testing stage, that the sterilization process is not sufficient in the sterilization stage, it is estimated that the sterilization process on the sets managed by the same job is not sufficient. Therefore, these set will not be used. For example, these sets are returned from the storage cabinet, and can be again subjected to the sterilization process.

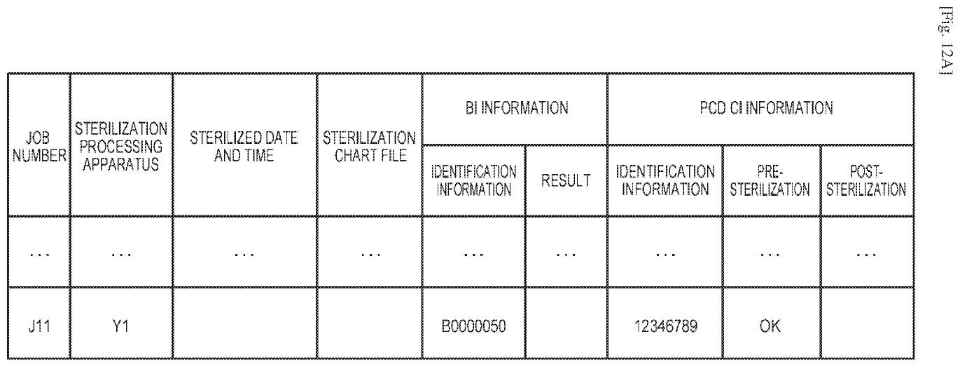

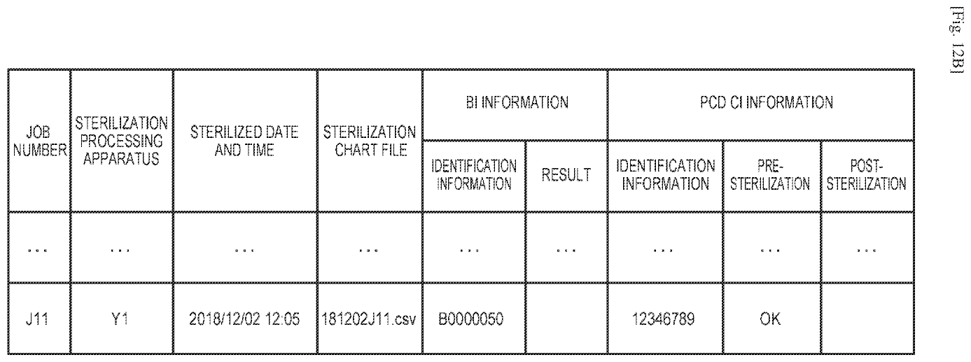

In the present embodiment, the server apparatus 14 includes a master information table and a job management table, as management information. In the following, each record in the job management table is denoted as a job record in order to distinguish it from a record in the master information table. Note that, the records in the master information table are simply denoted as records. FIGS. 11A and 11B show a master information table of the present embodiment. Information for specifying a job is recorded in a job number field. FIGS. 12A to 12C show a job management table of the present embodiment. Information for specifying a job is recorded in a job number field. Pieces of information similar to those in the master information table of the first embodiment are recorded in a sterilization processing apparatus field and a sterilization data and time field. Note that, in the present embodiment, information for specifying an individual sterilization processing apparatus 13 is recorded in the sterilization processing apparatus field. Information for specifying log information (log file) of the sterilization process performed by the sterilization processing apparatus 13 in the job is recorded in the sterilization chart file field. Information for specifying the BI that has been inserted into the sterilization processing apparatus 13 when the sterilization process was performed is recorded in an identification information subfield of a BI information field. A determination result of achievement degree of the sterilization process that has been determined by analyzing the BI that was used in the sterilization process is recorded in a result subfield of the BI information field. Information for specifying the PCD CI that was inserted into the sterilization processing apparatus 13 when the sterilization process was performed is recorded in an identification information subfield of a PCD CI information field. The determination result of an initial failure of the PCD CI is recorded in a pre-sterilization subfield. The determination result of achievement degree of the sterilization process that has been determined based on a measurement result of the PCD CI subjected to the sterilization process is recorded in a post-sterilization subfield.

For example, it is assumed that, when the contents in the master information table are as shown in FIG. 11A, the sterilization process is to be performed, in the sterilization stage, on two sets corresponding to records in numbers 101 and 102. of the master information table by a sterilization processing apparatus 13 of an individual Y1. In this case, an operator adds a new job record (job number J11) to the job management table as a recording target job record by operating the management system 100. Also, the operator records Y1 in the sterilization processing apparatus field of the recording target job record. Thereafter, the operator determines an initial failure by reading the PCD CI using the measurement apparatus 1. On this occasion, if the PCD CI has an initial failure, the management system 100 notifies the operator of a fact that a different PCD CI should be used. If the PCD CI has no initial failure, the management system 100 respectively records identification information of the PCD CI and “OK” in the identification information subfield and the pre-sterilization subfield of the PCD CI information field of the recording target, job record. Next, the operator inputs information to be recorded in the identification information subfield of the BI information field. FIG. 12A shows this state. Also, the operator records a job number J11 in the job number field of the records of numbers 101 and 102 as shown in FIG. 11B.

Also, after completing the sterilization process, the operator respectively records pieces of information in the sterilization data and time field and the sterilization chart file field of the recording target job record, as shown in FIG. 12B. Here, the configuration may be such that the pieces of information recorded here are acquired by the server apparatus 14 from the sterilization processing apparatus 13 via the network, for example. Alternatively, the configuration may also be such that the operator inputs the pieces of information into the management system 100 based on display or output contents of the sterilization processing apparatus 13. After the sterilization stage, the sets subjected to the sterilization process are transferred to the storing stage, and the PCD CI and the BI are transferred to the indicator testing stage. In the indicator testing stage, the PCD CI is measured by the measurement apparatus 1, and the determination result of the achievement degree of the sterilization process is recorded in the post-sterilization subfield of the PCD CI information field of the recording target job record. Meanwhile, with respect to the BI, the operator determines the achievement degree of the sterilization process following the determination procedure of BI, and records the determination result in the post-sterilization subfield of the BI information field.

In the present embodiment, if the determination result of the PCD CI or BI is “NG”, the sets subjected to the sterilization process in this job are prevented from being used. For example, as shown in FIG. 12C, if the determination result after measuring the PCD CI is “NG”, the management system 100 searches the job number J11, which is “NG”, from the job number field of the master information table. Next, the management system 100 extracts records in which J11 is recorded in the job number field of the master information table. In this example, the records of numbers 101 and 102 are extracted from the master information table in FIG. 11B. In this case, the management system 100 notifies the operator of the fact that the sets corresponding to the numbers 101 and 102 cannot be used in the operation stage, and need to be subjected to the sterilization process again.

Also, it is assumed that the sets corresponding to the records of numbers 101 and 102 have already been transferred to the operation stage, for example. In the operation stage, the CI enclosed in the set is measured in order to determine the achievement degree of the sterilization process. On this occasion, the records of numbers 101 and 102 in the master information table are extracted as the recording target record. On this occasion, the management system 100 confirms that the determination results of the PCD CI and the BI are “OK” based on the job number field of the recording target record and the job management table. Here, if one of the determination results of the PCD CI and the BI is “NG”, the management system 100 notifies an operator of the fact that the opened set should not be used and a different set should be used.

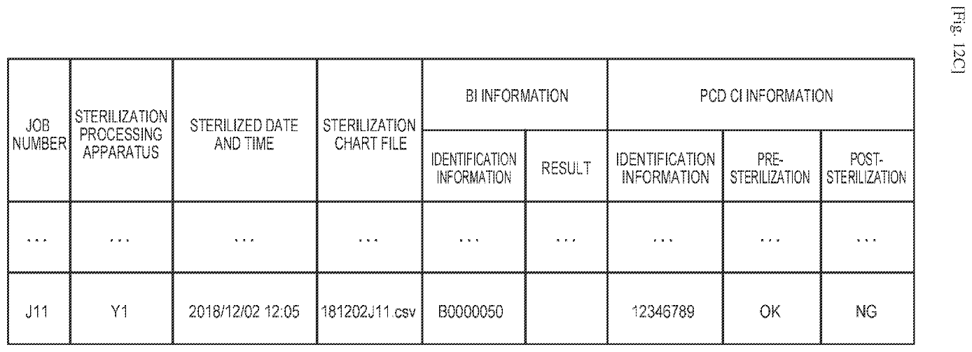

FIG. 13 shows an example of a graph that the management system 100 creates based on the job management table and is presented to a user. The graph shown in FIG. 13 shows monthly NG rate values of the PCD CI for each sterilization processing apparatus. The NG rate can be obtained by extracting monthly records for each sterilization processing apparatus, and calculating a ratio of the number of records in which the determination result of the PCD CI is NO relative to the number of extracted records. From FIG. 13, the NG rate of the sterilization processing apparatus of individual Y1 is 0 over a range from March to June. On the other hand, the NO rate of the sterilization processing apparatus of individual Y2 is 0 in March and April, but tends to increase in May and June. In such a case, it can be estimated that a change for causing degradation in the quality of the sterilization process has occurred in the sterilization processing apparatus of individual Y2. Therefore, it can be understood that, in order to reduce the NG rate, with respect to the sterilization processing apparatus of individual Y2, the portions that have been changed after May need to be reexamined and what has changed needs to be examined. With the analysis described above, the quality of the sterilization workflow can be improved. Note that a case where the determination of the PCD CI is “NG” has been described, but the same can apply to the case where the determination of the BI is “NO”.

### Third Embodiment

Next, a third embodiment will be described focusing on the difference from the second embodiment. In the storing stage, sets are stored in a packaged state, and a packaging member is opened in the operation stage. However, it is possible that the sterilized state of a sterilization target that has been stored over a long period of time in the storing stage has changed from the state immediately after the sterilization process. Therefore, in the present embodiment, a use-by date is set to a set subjected to the sterilization process. Also, if there is a set that has not been transferred to the operation stage even at a point in time close to the use-by date, the management system 100 notifies an operator of the fact that the operator should collect the set and subject the set to re-sterilization.

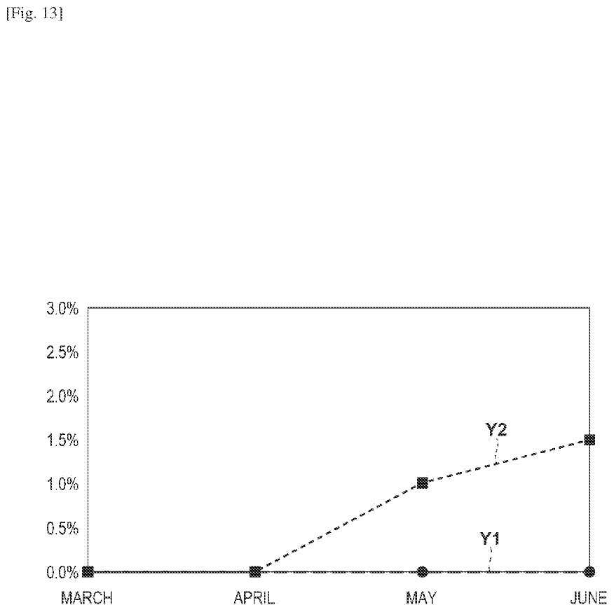

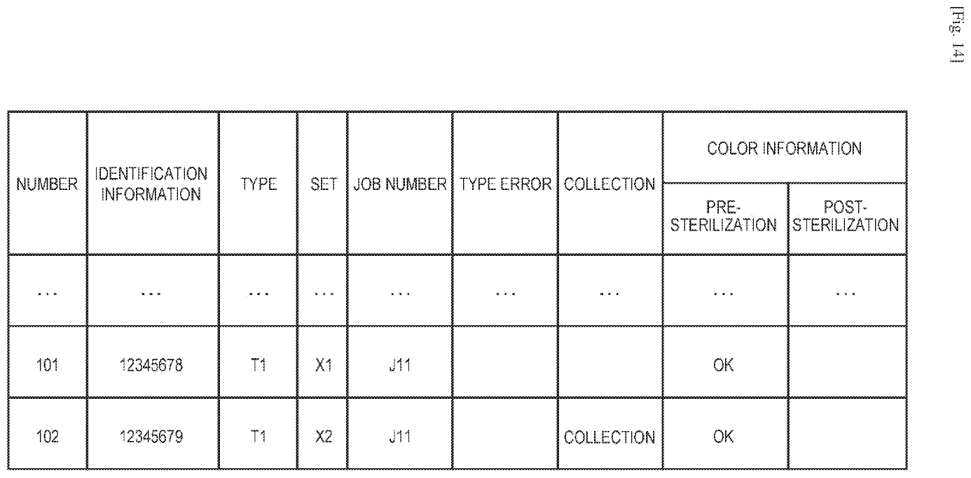

Therefore, in the present embodiment, a collection field is added to the master information table, as shown in FIG. 14. Also, a use-by date field and a number of stored sets field are added to the job management table, as shown in FIGS. 15A to 15C. Note that, in FIGS. 15A to 15C, only the job number field, the use-by date field, and the number of stored sets field are shown in order to simplify the display, and the other fields are omitted, because these fields are similar to those in FIGS. 12A to 12C. A use-by date of the set sterilized in a corresponding job is recorded in the use-by date field. The use-by date is set at a date after the sterilized date and time by a predetermined number of dates according to the sterilization program. The number of sets subjected to the sterilization process in the corresponding job is recorded in the number of stored sets field as shown in FIG. 15A. Note that the management system 100 can determine and record the value to be stored in the number of stored sets field by counting the number of job number fields, of the records in the master information table, that have the same value.

Also, the management system 100 records a use-by date in the use-by date field after the sterilization process, as shown in FIG. 15B. For example, the management system 100 can determine the use-by date from the date and time at which the sterilization process has been performed. Alternatively, the sterilization processing apparatus 13 determines the use-by date from the date and time at which the sterilization process has been performed, and the management system 100 can acquire the use-by date from the sterilization processing apparatus 13. Thereafter, the sets subjected to the sterilization process are stored in the storage cabinet. In the present embodiment, an operator reduces, every time bringing out sets from the storage cabinet in the delivery stage, the number in the number of stored sets field of the job number corresponding to the sets that have been brought out by the number of sets that have been brought out following the screen displayed in a PC 15. FIG. 15C shows a state in which, as a result of sets being brought out from the storage cabinet, the value in the number of stored sets field is updated to “5” from “7”.

Also, the management system 100 regularly searches the use-by date field and the number of stored sets field in the job management table, and determines whether or not a set is present with respect to which the number of dates until the use-by date is less than or equal to a predetermined period of time and the number of stored sets is greater than or equal to a threshold value. Note that the threshold value can be 1. If such sets are present, the management system 100 notifies an operator of the fact, and prompts the operator to collect the stored sets corresponding to the job and to perform the sterilization process again. The operator, upon collecting the sets following the notification from the management system 100, records “collected” in the collection field of the records corresponding to the collected sets, as shown in FIG. 14.

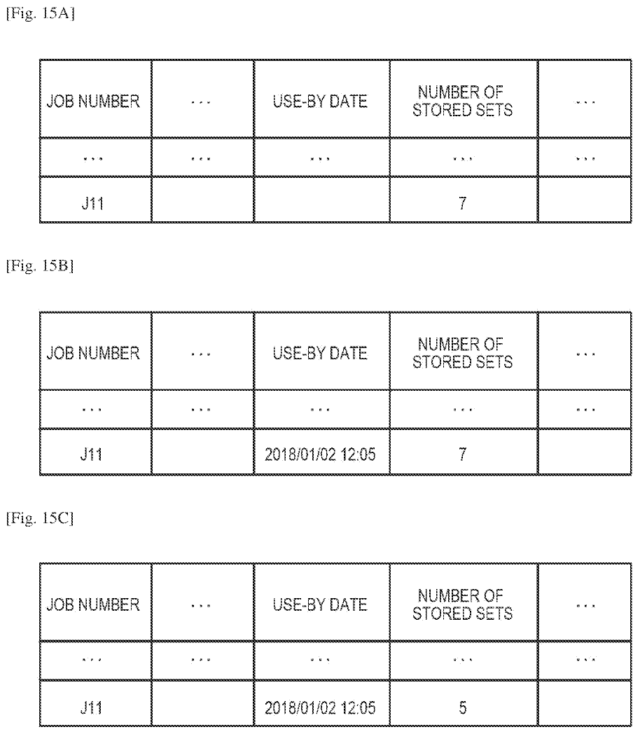

FIG. 16 shows an example of a graph that the management system 100 creates and presents to a user based on the job management table and the master information table of the present embodiment. FIG. 16 shows monthly collected numbers for each type of the set. From FIG. 16, it can be understood that the collected number of the sets of type X1 is low over a period from March to June. On the other hand, it can be understood that the collected number of the sets of type X2 tends to increase. In such a case, it is conceivable that use condition of the sets of type X2 has changed. Therefore, it can be understood that, in order to reduce the collected number of the sets of type X2, the change in the use condition of the sets of type X2 needs to be confirmed, and the number of stored sets of type X2 needs to be adjusted. As a result of the analysis described above, the improvement in quality of the sterilization workflow can be prompted.

### Fourth Embodiment

Next, a fourth embodiment will be described focusing on the difference from the third embodiment. When a set needed in an operation is not present in the storage cabinet, in the delivery stage, the sterilization process needs to be performed on the set in a short period of time. In order to perform sterilization process in a short period of time, infrequent work may need to be performed such as using a flash sterilization processing apparatus, which is different from a normally used apparatus, or performing, even with the same sterilization processing apparatus 13, the sterilization process with a different setting, which is different from the normal setting. Note that the setting different from the normal setting is to use temperature that is different from that in a normal case, to use a high-speed sterilization mode, or the like. Such an infrequent work induces a work error of an operator. Also, it is possible that a set needs to be used before obtaining testing results of the BI and the PCD CI. In the present embodiment, the frequency of urgent sterilization process is reduced. Therefore, the management system 100 manages a stock information table as the management information.

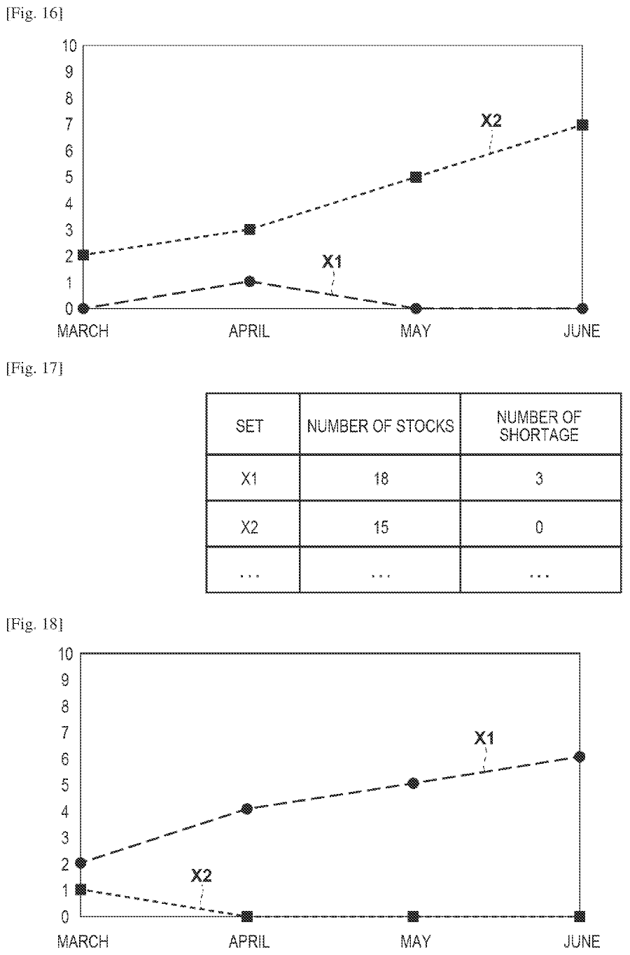

FIG. 17 shows an example of the stock information table. The stock information table includes a set field in which information for specifying the type of the set is recorded, a number of stocks field in which the number of the sets in the storage cabinet is recorded, and a number of shortages field in which the number of the stocked number having decreased below a predetermined number is recorded. The number of stocks field is increased when sets are stored in the storage cabinet, and is reduced when the sets are brought out from the storage cabinet. Note that updating of the number of stocks field is performed by an operator in accordance with a screen displayed in a PC 15. The management system 100 regularly searches the stock information table, and if the number of stocks is less than the predetermined number, prompts the operator to prioritize the sterilization process of the set by performing notification (shortage notification) to the operator. Also, the management system, upon performing a shortage notification, increments the value in the number of shortages field by 1. Note that the management system 100 separately manages the stock information table for each predetermined period of time, e.g. monthly, and records the monthly number of shortages.

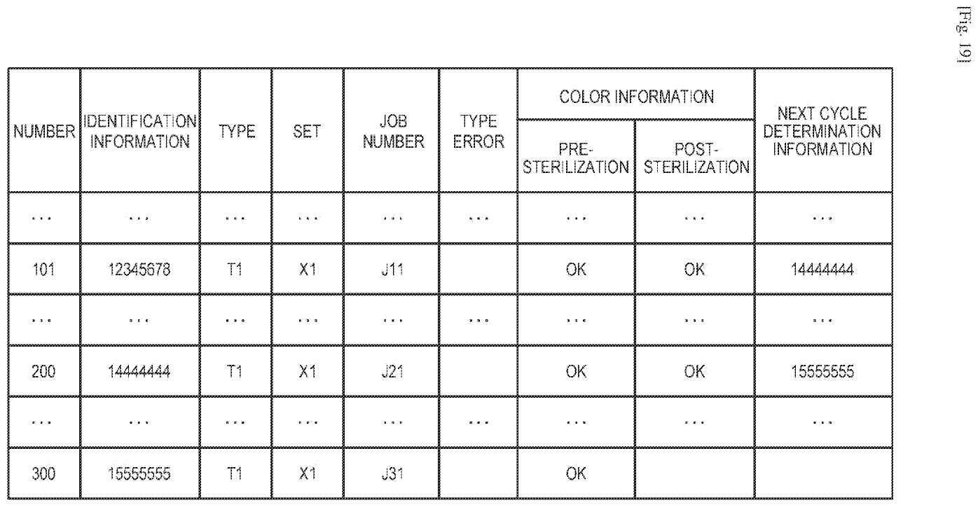

FIG. 18 shows an example of a graph that the management system 100 creates based on the stock information table and is presented to a user. FIG. 18 shows values of the monthly number of shortages for each type of the set. From FIG. 18, it can be understood that the number of times the stock number of the set of type X1 has decreased below the predetermined number tends to increase. On the other hand, it can be understood that the number of times the stock number of the set of type X2 has decreased below the predetermined number is small. In such a case, it is conceivable that use condition of the set of type X1 has changed. Therefore, from the graph shown in FIG. 18, it can be understood that, in order to optimize the number of stocks of each set, the change in the use condition of the set of type X1 needs to be confirmed, and the number of stocks needs to be adjusted. As a result of the analysis described above, the improvement in quality of the sterilization workflow can be prompted.

### Fifth Embodiment

Next, a fifth embodiment will be described focusing on the difference from the embodiments described above. As shown in FIG. 1, in the sterilization workflow, each set is circulated in a cycle of stages in which the set is transferred from the assembly stage to the cleaning stage via the stages, and after the cleaning stage, is again transferred to the assembly stage. In the present embodiment, for each set, identification information used in one cycle is associated with the identification information to be used in the next cycle.

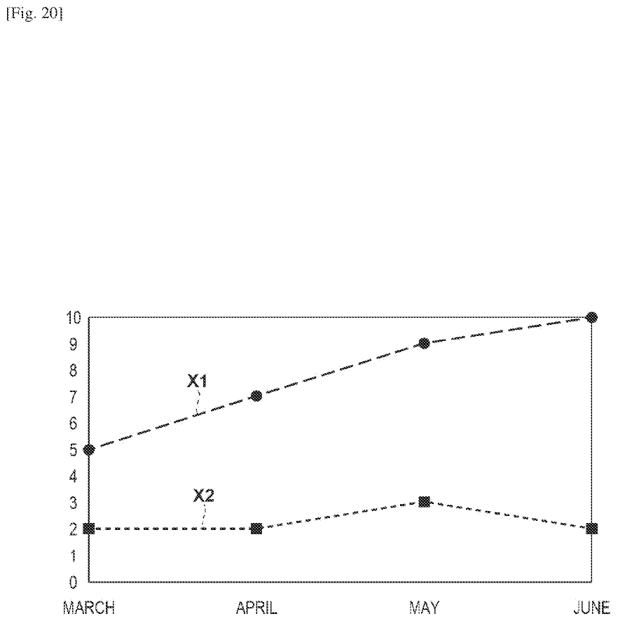

FIG. 19 shows a master information table in the present embodiment. In the present embodiment, a next cycle determination information field is provided in the master information table. In the next cycle determination information field of a record, the identification information of a CI enclosed, in the next packaging stage, in a set corresponding to the record is recorded. Note that in the present embodiment, with respect to a set corresponding to the record, the identification information of a CI enclosed in the next packaging stage is recorded, but the mode may be such that the identification information of a CI enclosed in the previous packaging stage is recorded. Note that each record in the master information table shows sterilization related information regarding one cycle of a set.

In the example in FIG. 19, the records of a number 101, a number 200, and a number 300 are records with respect to the same set, and therefore it can be understood that the set has been used three times. That is, from the management information shown in FIG. 19, the number of usages of each set can be grasped. As a result of grasping the number of usages of a set, and providing a limit on the number of usages, quality degradation of the set due to increase in number of usages can be suppressed. Note that, in the present embodiment, the number of usage of a set is managed by providing the next cycle determination information field, but the method of managing the number of usages of a set is not limited thereto, and the mode may be such that the number of usage is directly recorded in a record, for example. For example, it is assumed that the upper limit of the number of usages of a set of type X1 is three times. In this case, when the operation stage of the type X1 in a cycle corresponding to the number 300 is completed, the management system 100 determines the number of times used of the set. Then, if the number of times used is three times, which is the upper limit, as shown in the example in FIG. 19, the management system 100 notifies an operator to discard the set. Also, the management system 100 can manage the type of a set with respect to which discarding has been instructed, and the date thereof as discard information. The discard information is also a portion of the management information.

Note that quality degradation due to usage not only occurs in the sterilization target of a set, but also similarly occurs in a packaging member that can be re-used. Therefore, as a result of associating, with respect to a re-usable packaging member as well, the identification information used in the previous cycle with the identification information to be used in the next cycle, quality degradation due to usage can be avoided, similarly to the set. As a result of associating the identification information used in the previous cycle with the identification information to be used in the next cycle, not only the sets can be managed for each type, but the sets can be individually managed. Therefore, it is also possible to trace a usage history of a set individually. For example, it is possible to find out a trend by tracing past notification histories of individual sets, and associate information regarding operation and patient with respect to which the sets were used with replacement histories of sterilization targets of the sets, information regarding dropping of the sets, and the like.

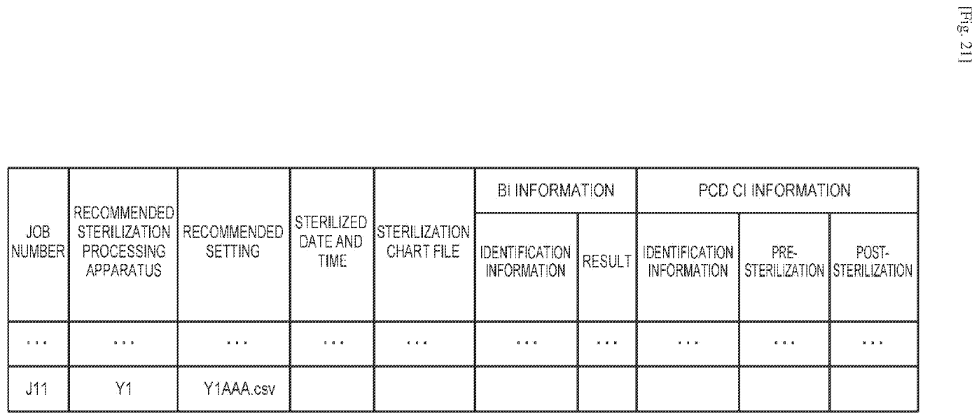

Also, in the present embodiment, the number of discards can be calculated by accumulating discard information of each set. FIG. 20 shows an example of a graph created based on the accumulated discard information. The graph shown in FIG. 20 shows monthly number of discards for each type of the set. From FIG. 20, it can be understood that the number of discards of the set of type X1 tends to increase. On the other hand, it can be understood that the number of discards of the set of type X2 remains at a small number. Therefore, from the graph in FIG. 20, it can be understood that the number of owned sets of type X1 needs to be adjusted, or the sets of type X1 need to be switched to sets of another type having a larger upper limit of the number of uses. As a result of the analysis described above, the quality of the sterilization workflow can be improved.

### Sixth Embodiment

Next, a sixth embodiment will be described focusing on the difference from the embodiments described above. In the present embodiment, a set that is scheduled to be used in an operation or the like can be reserved in advance. An operator inputs reservation information of a set in the management system 100 by operating a PC 15. In this case, the PC 15 functions as a reception unit of reservation information. The reservation information includes, with respect to each of the sets that are scheduled to be used, information regarding a type, number, and usage date and time and other pieces of additional information. With this, the management system 100 reserves sets of the specified type, of the sets stored in the storage cabinet, of an amount of the specified number. Here, if the sets of the specified type are not stored in the storage cabinet, or although the sets are stored, the number of the stored sets is less than the specified number, the management system 100 notifies an operator of information regarding the set that is not sufficient. With this, the risk factor for causing an urgent sterilization process can be reduced. Also, the number of owned sets can be optimized and the quality of the sterilization workflow can be improved by recording and analyzing the history of notification of the stock being insufficient.

### Seventh Embodiment

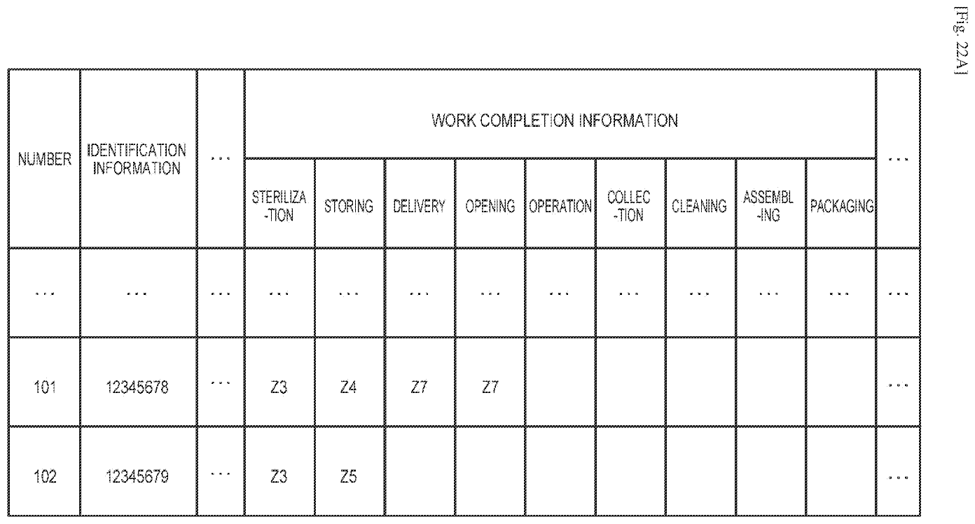

Next, a seventh embodiment will be described focusing on the difference from the embodiments described above. In the case of a set with respect to which the owned number is small, the necessity of the sterilization process in a short period of time increases. As described in the fourth embodiment, this fact may be a risk factor in a sterilization process work, and therefore, the operator needs to be notified of this fact. Therefore, in the present embodiment, a recommended setting field is added to the job management table, as shown in FIG. 21. Setting information indicating the recommended setting of the sterilization processing apparatus 13 when the set is subjected to the sterilization process is stored in the recommended setting field. Also, considering the possibility that an operator will use a wrong sterilization processing apparatus 13, the sterilization processing apparatus field is changed to a recommended sterilization processing apparatus field. The type of the sterilization processing apparatus 13 recommended with respect to the set and the number for specifying the apparatus are stored in the recommended sterilization processing apparatus field.

The management system 100 determines whether the sterilization process performed in the sterilization processing apparatus 13 is in accordance with the recommended setting indicated in the recommended setting field from the information in the sterilization chart file field. If the management system 100 determines that the sterilization process has not been performed in accordance with the recommended setting, the management system 100 can notify an operator of the fact that the sterilization process has not been performed in accordance with the recommended setting. Also, here, the configuration may be such that the reason why the work that is not recommended is performed or the like can be input as non-recommended work information. As a result of accumulating and analyzing such information, the type of sets on which sterilization in a short period of time is frequently performed, and the reason why the sterilization in a short period of time is performed can be specified. With this, if specific sets are used a plurality of times in a day, and the frequency of sterilization process in a short period of time is high, the risk of the occurrence of an infrequent work can be avoided by increasing the owned number of the sets.

### Eighth Embodiment

Next, an eighth embodiment will be described focusing on the difference from the embodiments described above. If an operator performs wrong work on a set in each stage in the sterilization workflow, the sterilized state of the set cannot be assured, and re-sterilization is needed. The work error is classified into “actual work/confirmation work itself having not been performed” and “actual work/confirmation work having not been properly performed”. One of the measures for reducing work errors is to notify the operator and/or a person concerned with the work of the fact that an error has occurred when the error has occurred. Also, specifying, by accumulating and analyzing error contents as data, an operator who makes an error frequently or a set with respect to which an error is likely to occur is also one measure for reducing an error. In the present embodiment, upon completing work determined in each stage of the sterilization workflow, the operator inputs “work completion information” into the management system 100. Set identification information, a type, actual work completion date and time, an actual operator name, confirmation work completion date and time, a confirmation operator name, a determination result of confirmation, measurement data when confirmation is performed, and the like can be recorded as the “work completion information”. Similarly to the first embodiment, the work completion information is stored in fields in the master information table suitable for the respective types of information.

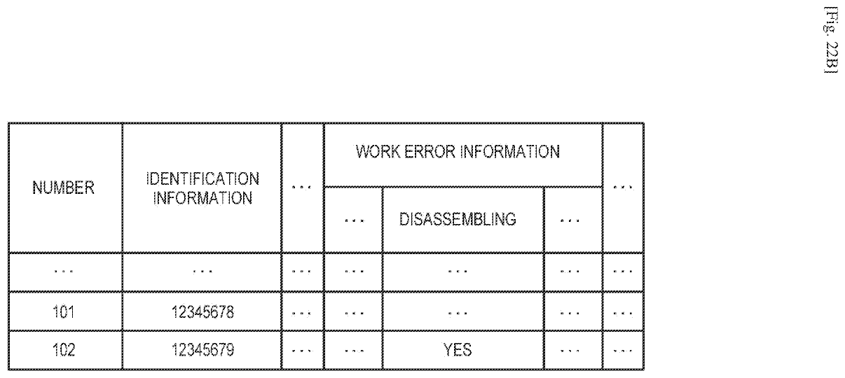

FIG. 22A shows an example of a master information table for preventing the occurrence of “actual work/confirmation work itself having not been performed”. According to FIG. 22A, information for specifying an actual operator is stored in a subfield of each stage as the “work completion information”. In FIG. 22A, if the opening stage is tried to be performed on a set corresponding to the record of number 102, for example, because the record of operator information is not present in a delivery subfield, the management system 100 notifies of this fact. In this way, if work completion information of a downstream stage is input in a state in which at least one of pieces of work completion information of upstream stages has not been input, the management system 100 notifies of the fact that work completion information of an upstream stage has not been input. Moreover, a scheduled time at which each stage is to be performed on a set can also be input to the management system 100. The management system 100 records, in association with each subfield of the work completion information field of a record corresponding to the set, the scheduled time at which the corresponding stage is to be performed. Also, if the work completion information has not been registered even if a certain time has passed since the scheduled time of the work, the management system 100 can notify the user of this fact.

FIG. 22B shows a master information table for preventing the occurrence of “actual work having not been properly performed”. In FIG. 22B, a work error information field is added, in addition to the work completion information field. In the work error information field, a subfield corresponding to work in each stage is provided, and if an error is found out later, “YES” is input to a subfield corresponding to work with respect to which an error has been found out. Note that an example of “actual work having not been properly performed” is performing cleaning in a state in which a set or a sterilization target in the set has not been properly disassembled in collection and cleaning stages. Also, the example includes not properly assembling a sterilization target or a set in assembling and packaging stages. Moreover, the example includes a case in which, in the packaging stage, a CI of a wrong type is enclosed in the packaging member, a CI itself has not been enclosed in the packaging member, or identification information has been forgotten to be attached to an exterior face of a packaging member. Furthermore, the example includes a case in which, in the sterilization stage, a set is inserted into a sterilization processing apparatus 13 of a wrong type, a PCD CI of a wrong type is inserted into a sterilization processing apparatus 13, or a PCD CI itself has been forgotten to be inserted into a sterilization processing apparatus 13. In the present embodiment, if it is found out, in a later stage, that work in a previous stage has not been properly performed, “YES” is input to a corresponding work subfield. Also, with this, the management system 100 notifies a related operator of this fact.

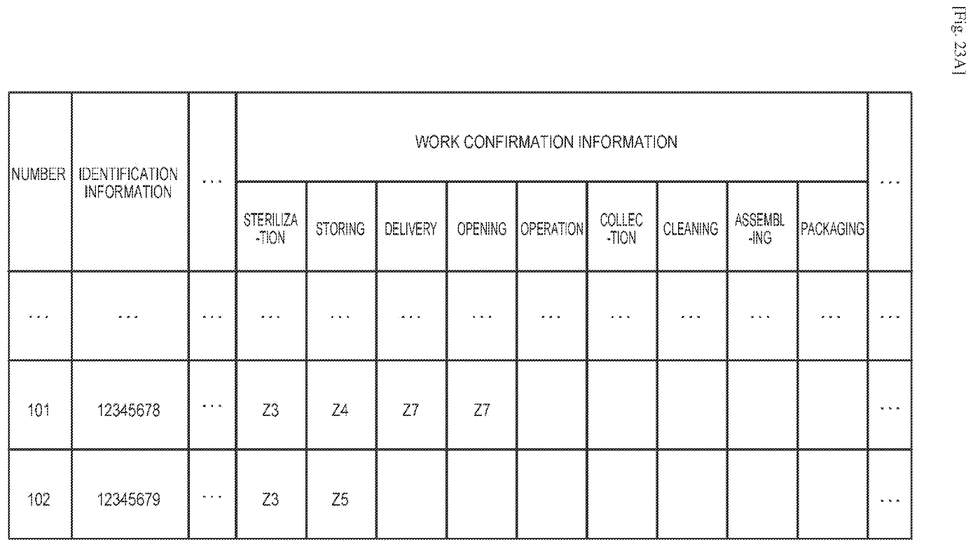

FIG. 23A shows a master information table for preventing the occurrence of “confirmation operation having not been properly performed”. According to FIG. 23A, information for specifying a confirmer is stored in a subfield of each stage in addition to the “task completion information” in FIG. 22A. The confirmer, upon confirming completion of the stage, inputs information for specifying the confirmer into the management system 100. An example of “confirmation operation having not, been properly performed” includes a case of not noticing damage of a packaging member that packages a set. Also, as shown in FIG. 23B, a configuration may also be adopted in which a plurality of confirmers perform confirmation in one stage. In FIG. 23B, two confirmers perform confirmation in each stage, and information for specifying each confirmer and information regarding whether the confirmation result by each confirmer is OK or NG are recorded in the master information table. For example, if at least one confirmer determined as NG, the management system 100 notifies an operator or a person concerned of this fact.

Also, as a result of recording and accumulating notifications of errors or the like, an operator who frequently made an error can be specified, or a set with respect to which an error is likely to occur can be specified. With this, a measure can be taken such as retraining an operator who frequently made an error again, or a detailed manual is attached to a set with respect to which an error is likely to occur.

### Ninth Embodiment

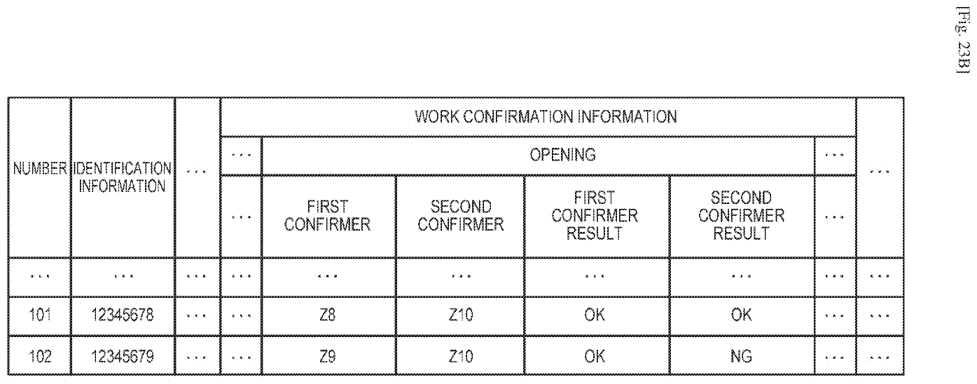

Next, a ninth embodiment will be described focusing on the difference from the embodiments described above. The degree of importance of risk factors that cause quality degradation of the sterilization process are not the same. For example, if a stocked set immediately before a use-by date, which is described in the third embodiment, is actually used after the use-by date, it can be a serious risk factor. However, the reduction of stocked sets, which is described in the fourth embodiment, even if urgent sterilization process is performed due to stock shortage, is not a big problem as long as the sterilization process is properly performed. That is, the management of use-by dates of sets is more important than managing the stocks. In the present embodiment, the quality of the sterilization workflow is improved by performing analysis by weighing risk factors. For example, in the third and fourth embodiments, the number obtained by adding the number obtained by multiplying the number of records in which “collected” is recorded in the collection field, which is more important, by 10 and the number obtained by multiplying the number of shortages, which is relatively less important, by 2 is defined as a stock risk index. FIG. 24 shows monthly stock risk index of each of sets of types X1 and X2. From FIG. 24, it can be understood that optimization of the number of stored sets of type X2 whose stock risk index is increasing more is prioritized.

Functional Blocks of Management System

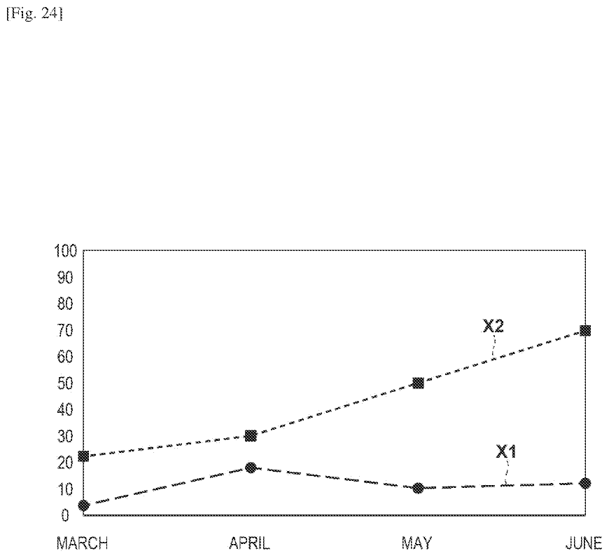

FIG. 25 is a functional block diagram of the management system 100 shown in FIG. 2. A retention unit 103 corresponds to a storage unit such as a hard disk of the server apparatus 14, and retains management information including sterilization record information. An input/output unit 106 corresponds to a PC 15, for example, accepts an input of information that a user records in the management information, and outputs information to be presented to a user. Also, the input/output unit 106 corresponds to the measurement apparatus 1 and a PC 15, measures the color value of a discoloration area of a CI, and reads identification information of the CI. The input/output unit 106 outputs information input by a user, the measured color value of a CI, and identification information of the CI that has been read to the management unit 104. Moreover, the input/output unit 106 outputs log information or the like acquired from a sterilization processing apparatus 13 to the management unit 104. Note that, when the identification information of a CI is not used as the identification information of a set, and identification information is attached to a packaging member of the set, for example, the input/output unit 106 includes an IC reader or a bar-code reader, and outputs read identification information to the management unit 104. The management unit 104 corresponds to a processor of the server apparatus 14 or a PC 15, and performs addition of information to the management information, update of the management information, and deletion of unnecessary information recorded in the management information, as necessary, based on the information from the input/output unit 106.

A notification unit 102 performs notification to a user based on management information. Note that the notification performed by the notification unit 102 includes a notification indicating that work relating to the workflow is not in a predetermined state. For example, the notification unit 102 notifies of an error in work regarding the workflow, as work being not in a predetermined state. Moreover, the notification made by the notification unit 102 includes a notification for preventing work relating to the workflow from entering a predetermined state. For example, the notification unit 102 can make a notification for preventing an error in work relating to the workflow. For example, the notification described in the first, second, seventh, and eighth embodiments is a notification indicating that the work is not in a predetermined state. On the other hand, the notification described in the third to sixth embodiments is a notification for preventing work relating to the workflow from not entering a predetermined state. In other words, the notification unit 102 makes, to a user based on the management information, a notification that an inappropriate state has been entered regarding the workflow, or a notification for preventing entering an inappropriate state regarding the workflow. For example, the notification of the first, second, seventh, and eighth embodiments is a notification that the workflow is inappropriate. On the other hand, the notification of the third to sixth embodiments is a notification of preventing the workflow from entering an inappropriate state. The information generation unit 105 performs analysis as described in the embodiments described above based on the management information, and presents the analysis result to a user via the input/output unit 106. Specifically, the management unit 104, upon making a notification described above, records the contents of the notification in the management information. Also, the information generation unit 105 analyzes the management information in order to suppress inappropriate states from occurring, and presents the analysis result to a user.

Functional Blocks of Information Processing Apparatus

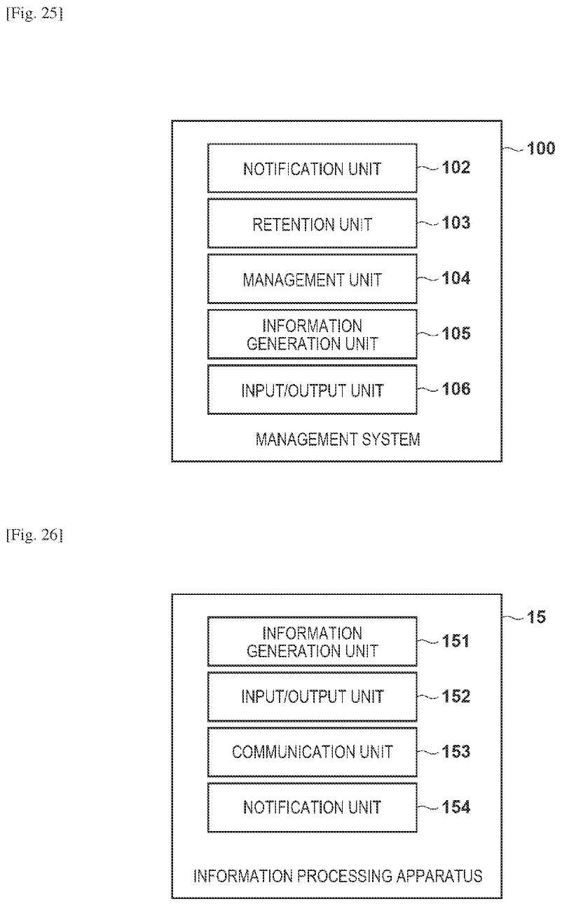

Note that each PC 15 in FIG. 2 is also an information processing apparatus that makes a notification to a user based on the sterilization record information of the management information, and generates pieces of information described in the embodiments described above based on the management information. FIG. 26 is a functional block diagram of a PC 15 (information processing apparatus). An input/output unit 152 corresponds to a keyboard, a mouse, a display, a printer, and a storage apparatus of the PC 15, receives operational inputs from a user, and outputs information to be presented to the user. A communication unit 153 is an accessing unit for acquiring management information retained by the server apparatus 14 by accessing the server apparatus 14. A notification unit 154 and an information generation unit 151 are similar to the notification unit 102 and the information generation unit 105 described in FIG. 25. Note that, the notification unit 154, upon performing a notification described above, notifies the server apparatus 14 of the notified contents for causing the server apparatus 14 to record the contents in the management information.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform. the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-034744, filed on Feb. 27, 2019, which is hereby incorporated by reference herein in its entirety.

1. A management system that manages a workflow relating to sterilization of a sterilization target,
wherein the workflow includes a plurality of stages that includes at least one of a packaging stage in which a set is formed by packaging one or more of the sterilization targets and an indicator, a sterilization stage of subjecting the set to a sterilization process by a sterilization processing apparatus, and a using stage of using the one or more of the sterilization targets included in the set by determining an achievement degree of the sterilization process performed on the set using the indicator of the set,
wherein the indicator includes a discoloration area a color of which changes according to the achievement degree of the sterilization process, and wherein the management system includes:
a management unit configured to manage management information of the workflow including sterilization related information of the set that is associated with identification information of the set, wherein the sterilization related information of the set includes a determination result of the achievement degree of the sterilization process determined using the indicator, and wherein the stages of the workflow that have been completed with respect to the set can be determined from the sterilization related information of the set;
an input unit configured to input the sterilization related information of the set; and
a notification unit configured to perform, to a user based on the management information, a notification that work related to the workflow is not in a predetermined state, or a notification for preventing work related to the workflow from entering a predetermined state.

2. The management system according to claim 1, wherein the input unit includes a reading unit configured to read the identification information of the set.

3. The management system according to claim 1, wherein the input unit includes:
a measurement unit configured to measure a color of the discoloration area of the indicator; and
a determination unit configured to determine the achievement degree of the sterilization process based on a measurement result of the measurement unit.

4. The management system according to claim 1, wherein the identification information of the set is identification information given to the indicator to be enclosed in the set.

5. The management system according to claim 4, wherein the notification unit determines a number of times used of the set, and if the number of times used of the set reaches an upper limit of the number of times used of the set, performs a first notification for indicating the user of discarding of the set.

6. The management system according to claim 5, wherein the management unit records cycle determination information indicating a relationship between the identification information given to the indicator enclosed in the set in the packaging stage and the identification information given to the indicator enclosed in the set in the packaging stage of a previous time or a next time with respect to the set as the sterilization related information of the set, and uses the cycle determination information for determining the number of times used of the set.

7. The management system according to claim 1,
wherein the management unit records information indicating a type of the set, a type of the indicator enclosed in the set, and a type of the sterilization processing apparatus to be used in sterilization process of the set as the sterilization related information of the set, and
wherein the notification unit retains determination information indicating a combination between a type of the set, a type of the indicator, and a type of the sterilization processing apparatus to be used in sterilization process of the set, and
wherein, if the combination of the type of the set, the type of the indicator enclosed in the set, and the type of the sterilization processing apparatus to be used in the sterilization process of the set that is indicated by sterilization related information of the set does not match the combination indicated by the determination information, the notification unit performs a notification for notifying the user of the fact of not matching.

8. The management system according to claim 1,
wherein the management unit records information indicating a log output by the sterilization processing apparatus used in the sterilization process of the set as the sterilization related information of the set, and
wherein the notification unit retains setting information indicating a recommended setting of the sterilization processing apparatus corresponding to a type of the set, determines whether the sterilization process performed on the set has been performed in the recommended setting based on the log indicated by the sterilization related information of the set, and upon determining that the sterilization process performed on the set has not been performed in the recommended setting, performs a notification for notifying the user of a fact that the sterilization process performed on the set has not been performed in the recommended setting.

9. The management system according to claim 1,
wherein the indicator is a first indicator,
wherein the management unit records a determination result of the achievement degree of the sterilization process obtained by using a second indicator arranged inside the sterilization processing apparatus along with the set as the sterilization related information of the set, and
wherein, if the sterilization related information of the set indicates that the achievement degree of the sterilization process has not reached a predetermined achievement degree, which is indicated by the determination result obtained by using the second indicator, the notification unit performs a notification for notifying the user of a fact that the set cannot be used in the using stage.

10. The management system according to claim 1, wherein the management unit, upon receiving an input of date and time at which the sterilization process has been performed on the set, sets a use-by date until which the set can be used in the using stage based on the input date and time, and records the use-by date as the sterilization related information of the set.

11. The management system according to claim 10, wherein the notification unit, if the set is present, of the stored sets that have been subjected to the sterilization stage and have not been subjected to the using stage, with respect to which a period until the use-by date is less than or equal to a predetermined period, which is determined based on the sterilization related information of the set, performs a notification for notifying the user of a fact that the set with respect to which a period until the use-by date is less than or equal to the predetermined period is present.

12. The management system according to claim 10, wherein the notification unit determines a number of the sets, of the stored sets that have been subjected to the sterilization stage and have not been subjected to the using stage, with respect to which a period until the use-by date is less than or equal to a predetermined period, based on the sterilization related information of each set, and if the number of sets with respect to which the period until the use-by date is less than or equal to the predetermined period is larger than a predetermined number, performs a notification for notifying the user of a fact that the number of sets with respect to which the period until the use-by date is less than or equal to the predetermined period is larger than the predetermined number.

13. The management system according to claim 1, wherein the notification unit obtains a number of the stored sets that have been subjected to the sterilization stage and have not been subjected to the using stage for each type of the set based on the sterilization related information of each set, and if a type of the set with respect to which the number of the stored sets is less than a predetermined number is present, performs a notification for notifying the user of a fact that a type of the set with respect to which the number of the stored sets is less than the predetermined number is present.

14. The management system according to claim 13, further comprising a reception unit configured to receiving reservation of the set to be used in the using stage,
wherein the notification unit, if the reservation cannot be satisfied with the stored sets, performs a notification for notifying the user of a fact that the reservation cannot be satisfied with the stored sets.

15. The management system according to claim 1, wherein the management unit records information indicating an operator of each stage in the workflow as the sterilization related information of the set, and
wherein the notification unit, when information indicating an operator of a stage of the workflow is recorded, if information indicating an operator of a stage upstream of the stage in the workflow is not recorded in the sterilization related information of the set, performs a notification notifying the user of a fact that information indicating an operator of a stage upstream of the stage is not recorded.

16. The management system according to claim 1, wherein the management unit records information indicating a confirmer of work of each stage of the workflow as the sterilization related information of the set, and
wherein the notification unit, when information indicating a confirmer of work of a stage of the workflow is recorded, if information indicating a confirmer of work of a stage upstream of the stage in the workflow is not recorded in the sterilization related information of the set, performs a notification notifying the user of a fact that information indicating a confirmer of work of a stage upstream of the stage is not recorded.

17. The management system according to claim 1, wherein the management unit records, with respect to at least one stage of the workflow, information indicating a plurality of confirmers of a work, and respective confirmation results by the plurality of confirmers as the sterilization related information of the set, and
wherein the notification unit, if at least one of confirmation results by the plurality of confirmers with respect to the at least one stage is “NG”, performs a notification notifying the user of a fact that the at least one stage is “NG”.

18. The management system according to claim 1, wherein the management unit, upon performing the notification, records the notified contents to the management information, and
wherein the management system further includes a presenting unit configured to present an analysis result of the management information for suppressing work relating to the workflow from entering the predetermined state.

19. The management system according to claim 7, wherein the management unit records information indicating an operator that has input information indicating a type of the set, a type of the indicator enclosed in the set, and a type of the sterilization processing apparatus to be used in the sterilization process of the set, and information indicating whether or not a combination that does not match a combination indicated by the determination information has been input, as the sterilization related information of the set, and
wherein the management system further includes a presenting unit configured to present the user of a change in a number of times, for each operator, that a combination that does not match a combination indicated by the determination information has been input based on the sterilization related information of the set.

20. The management system according to claim 5, wherein the management unit records information indicating a set with respect to which the first notification has been made, and
wherein the management system further includes a presenting unit configured to present the user of a change in a number of sets, for each type of the set, that the notification unit has notified of discarding based on the management information.

21. The management system according to claim 12, wherein the management unit records, to the sterilization related information of the set with respect to which the notification has been made, a fact that the notification has been made with respect to the set, and
wherein the management system further includes a presenting unit configured to present the user of a change in a number of sets, for each type of the set, with respect to which the notification has been made based on sterilization related information of the set.

22. The management system according to claim 13, wherein the management unit records, for each type of the set, a number of times that the notification has been made for each predetermined period to the management information, and
wherein the management system further includes a presenting unit configured to present the user of a change in a number of times, for each type of the set, that the notification has been made based on the management information.

23. The management system according to claim 10, wherein the management unit, if the set is present, of the stored sets that have been subjected to the sterilization stage and have not been subjected to the using stage, with respect to which a period until the use-by date is less than or equal to a predetermined period, records a fact that a period until the use-by date is less than or equal to a predetermined period to the sterilization related information of the set, and record a number of times, for each type of the set, that the number of the stored sets has decreased below a predetermined number, for each predetermined period, to the management information, and
wherein the management system obtains, for each type of the set, a number of the sets with respect to which a period until the use-by date has become less than or equal to a predetermined period, for each of the predetermined periods, based on management information of the set, obtains a change in a number that is obtained, for each type of the set, by adding a number of sets with respect to which a period until the use-by date has become less than or equal to a predetermined period and a number of times that the number of stored sets has decreased below a predetermined number by applying different weights, and further includes a presenting unit configured to present to the user the change in the obtained number.

24. An information processing apparatus comprising:
one or more processors; and
one or more memories including a program,
wherein the program, when executed by the one or more processors, causes the one or more processors to perform operations including:
accessing an apparatus having management information regarding a workflow including a plurality of stages that includes at least one of a packaging stage in which a set is formed by packaging one or more of sterilization targets and an indicator, a sterilization stage of subjecting the set to a sterilization process by a sterilization processing apparatus, and a using stage of using the one or more of the sterilization targets included in the set by determining an achievement degree of the sterilization process performed on the set using the indicator of the set, wherein the management information includes sterilization related information of the set that is associated with identification information of the set, the sterilization related information of the set includes a determination result of the achievement degree of the sterilization process determined using the indicator, and wherein which of the packaging stage, the sterilization stage, and the using stage has been completed with respect to the set can be determined from the sterilization related information of the set; and
performing, to a user based on the management information, a notification that work related to the workflow is not in a predetermined state, or a notification for preventing work related to the workflow from entering a predetermined state.

25. The information processing apparatus according to claim 24,
wherein the operations further include:
notifying, upon performing the notification, the apparatus having the management information of notified contents so that the contents are recorded in the management information, and
presenting an analysis result of the management information for suppressing work relating to the workflow from entering the predetermined state.

26. A non-transitory computer readable storage medium storing a program that, when executed by one or more processors in an apparatus including the one or more processors, causes the apparatus to perform operations including:
accessing an apparatus having management information regarding a workflow including a plurality of stages that includes at least one of a packaging stage in which a set is formed by packaging one or more of sterilization targets and an indicator, a sterilization stage of subjecting the set to a sterilization process by a sterilization processing apparatus, and a using stage of using the one or more of the sterilization targets included in the set by determining an achievement degree of the sterilization process performed on the set using the indicator of the set, wherein the management information includes sterilization related information of the set that is associated with identification information of the set, the sterilization related information of the set includes a determination result of the achievement degree of the sterilization process determined using the indicator, and wherein which of the packaging stage, the sterilization stage, and the using stage has been completed with respect to the set can be determined from the sterilization related information of the set and
performing, to a user based on the management information, a notification that work related to the workflow is not in a predetermined state, or a notification for preventing work related to the workflow from entering a predetermined state.

27. A management method in a management system that manages a workflow relating to sterilization of a sterilization target
wherein the workflow includes a plurality of stages that includes at least one of a packaging stage in which a set is formed by packaging one or more of the sterilization targets and an indicator, a sterilization stage of subjecting the set to a sterilization process by a sterilization processing apparatus, and a using stage of using the one or more of the sterilization targets included in the set by determining an achievement degree of the sterilization process performed on the set using the indicator of the set,
wherein the indicator includes a discoloration area a color of which changes according to the achievement degree of the sterilization process, and
wherein the management method includes:
managing, by recording, management information of the workflow including sterilization related information of the set that is associated with identification information of the set, wherein the sterilization related information of the set includes a determination result of the achievement degree of the sterilization process determined using the indicator, and wherein the stages of the workflow that have been completed with respect to the set can be determined from the sterilization related information of the set; and
notifying a user, based on the management information, of a fact that work related to the workflow is not in a predetermined state, or of a notification for preventing work related to the workflow from entering a predetermined state.
